(19) United States
(12) Patent Application Publication
(10) Pub. No.: US 2012/0183443 A1
(43) Pub. Date: Jul. 19, 2012

(54) AIR PURIFICATION DEVICE

(76) Inventor: John HURLEY, Toronto (CA)

(21) Appl. No.: 13/349,643

(22) Filed: Jan. 13, 2012

Related U.S. Application Data
(60) Provisional application No. 61/432,744, filed on Jan. 14, 2011.

(51) Int. Cl. A61L 9/20

(52) U.S. Cl. 422/121; 96/224

(57) ABSTRACT

Disclosed herein is a device for the removal of biological and chemical contaminants from an air current The apparatus comprises a) a housing that defines an interior chamber, the housing having an inlet and an outlet; b) one or more ultraviolet emitting light sources positioned within the interior chamber; and c) one or more baffles providing resistance to airflow, wherein air enters the chamber through the inlet, travels through the inner chamber, and exits via the outlet. In addition, the apparatus includes a photocatalyst.

### TECHNICAL FIELD

The present device relates to the field of air purification, and specifically to the removal of biological and chemical contaminants.

### BACKGROUND

Indoor air quality (IAQ) is particularly important as it has a direct impact on the health, safety and general comfort of the inhabitants of a building. The IAQ is affected by a multitude of factors, such as biological contaminants, e.g. bacteria, molds and viruses; chemical contaminants, e.g. volatile organic compounds (VOCs), carbon monoxide and formaldehyde; and particulate matter, e.g. dust and antigenic particles. When IAQ levels in a building are insufficiently dealt with and reach low levels, the inhabitants may develop an ailment that is directly associated with one or more of the contaminants, or experience various symptoms associated with sick building syndrome.

A portion of the increasing occurrence of decreased IAQ can be attributed to the advances in construction technologies, where buildings are designed and built to be essentially air tight. In such a construction, the internal air continuously re-circulates with little to no turnover, and therefore, there are minimal opportunities to dilute or eliminate potential contaminants. However, advances in air purification technology have also been made, and current air purifiers are becoming increasingly capable of capturing and/or eliminating a greater number of contaminants.

There exist several different processes of varying effectiveness that are used in air purifiers to purify air. Different processes are typically directed toward different contaminants, so it is typically advantageous to use more than one process in an air purifier.

Particulate filters that trap airborne particles by size exclusion, such as high efficiency particulate air (HEPA) filters, are the most common. Air is forced through the filter and particles are physically captured and removed. These types of filters are typically only effective at capturing particles that are 0.3 microns or larger, and therefore, do very little to decrease the levels of most biological and/or chemical contaminants.

One option to address biological contaminants is ultraviolet germicidal irradiation (UVGI), which is becoming more frequently utilized to sterilize air, such as in heating, ventilating and air-conditioning (HVAC) systems. In UVGI, circulating air is exposed to ultraviolet light of sufficient wavelength, for example 254 nm, which is mutagenic to microbial DNA. Microbial growth and reproduction is subsequently reduced or inhibited altogether.

Decreasing chemical contaminants in circulating indoor air is currently being addressed by the emerging technology, photocatalytic oxidation (PCO). Photocatalytic oxidation comprises exposing a photocatalyst, such as titanium dioxide (TiO₂), to light of an appropriate wavelength. Exposure of the photocatalyst to the light photons causes the formation of reactive oxygen species (ROS), such as hydroxyl radicals and superoxide. The ROS react with many chemical contaminants to form harmless byproducts, such as water and carbon dioxide. ROS have also been shown to have detrimental effects through various mechanisms on numerous microbes. This technology is harnessed, for example, by installing an apparatus that has a light source (such as a UV lamp) in close proximity to a photocatalyst, in an HVAC system. The circulating air is then exposed to the generated ROS, thereby decreasing the overall chemical and/or biological contamination, which increases the IAQ.

One potential drawback, which is applicable to both UVGI and PCO technology, occurs when the circulating air is moving at a rapid rate, and therefore is only exposed to the UV light and ROS for a short period of time. In such a scenario, microbial mutagenesis and/or complete breakdown of chemical contaminants does not always occur. In the case of PCO, this can result instead in the formation of chemical intermediate byproducts, which will usually circulate for at least another pass throughout the building before it has the opportunity to broken down further. Depending upon the starting material, breakdown to an intermediate may be sufficient to increase the overall IAQ, however, in some instances, the intermediates may be more harmful than the starting material, and therefore, the IAQ is actually lowered. Accordingly, it is desirable to increase the exposure time of the circulating air to the ROS and to the UV light.

As noted above, multiple types of filtering technologies are often coupled together to increase the efficiency of the air purification.

For example, U.S. Patent Publication No. 2004/0041564 discloses a method and system for improving air quality where combinations of air purification techniques are utilized. The system includes a conduit having at least one of an ultraviolet light source, a humidity control unit, a turbulator, a filter, a blower and a ventilator.

U.S. Patent Publication No. 2005/0063881 discloses a purification system having a treatment chamber with an inlet and an outlet. A radiation source and a photocatalytic reactor are provided within the chamber. The interior surface of the treatment chamber is reflective for radiation emitted by the radiation source. Baffles are provided at the inlet of the chamber.

Sanuvox™ (Montreal, Canada) offers for sale a variety of air purifiers for installation into an HVAC system, such as the R4000GX In-Duct UV Air Purifier. This device has a chamber with an inlet and an outlet, where a turbulator is positioned at the inlet. A split U-shaped lamp that emits UV-V light at one end and UV-C light at the opposing end is placed along the length of the chamber. The interior surface of the chamber is reflective to direct and concentrate the ultraviolet light.

### SUMMARY

According to one aspect, there is provided an air purification apparatus comprising: a) a housing that defines an interior chamber, the housing having an inlet and an outlet; b) one or more ultraviolet emitting light sources positioned within the interior chamber; and c) one or more baffles providing resistance to airflow, wherein air enters the chamber through the inlet, travels through the inner chamber, and exits via the outlet.

The apparatus can further include a photocatalyst, such as, but not limited to, TiO₂. In one embodiment, at least a portion of the interior of the housing and/or the plurality of baffles is coated with a photocatalyst. In the apparatus, each ultraviolet emitting light source can be independently either UV-V light or UV-C light. Furthermore, the number of ultraviolet emitting light sources can be one or two. In addition, a portion of an inner surface of the housing can comprise a reflective material. Where the apparatus includes one baffle, the baffle is continuous throughout a length of the chamber. In such a case, the baffle can have a spiral or coil-like shape. In addition, the apparatus can include a sensor for measuring the concentration level of one or more contaminants in the air.

In another aspect, there is provide an air purification apparatus comprising: a) a housing that defines an interior chamber, the housing having an inlet and an outlet; b) one or more ultraviolet emitting light source receiving members, each receiving member positioned within the inner chamber; and c) one or more baffles providing resistance to airflow, wherein air enters the chamber through the inlet, travels through the inner chamber, and exits via the outlet.

The apparatus can further include a photocatalyst, such as, but not limited to, TiO₂. In one embodiment, at least a portion of the interior of the housing and/or the plurality of baffles is coated with a photocatalyst. In the apparatus, each receiving member can independently receive a ultraviolet emitting light source that is either UV-V light or UV-C light. Furthermore, the number of ultraviolet emitting light sources can be one or two. In addition, a portion of an inner surface of the housing can comprise a reflective material. Where the apparatus includes one baffle, the baffle is continuous throughout a length of the chamber. In such a case, the baffle can have a spiral or coil-like shape. In addition, the apparatus can include a sensor for measuring the concentration level of one or more contaminants in the air.

In yet another aspect, there is provided an air purification apparatus comprising: a) a housing that defines an interior chamber, the housing having an inlet and an outlet; and b) one or more ultraviolet emitting light sources positioned within the inner chamber, with at least one ultraviolet emitting light source serving as a baffle to provide resistance to airflow through the interior chamber, wherein air enters the chamber through the inlet, travels through the inner chamber, and exits via the outlet.

The apparatus can further include a photocatalyst, such as, but not limited to, TiO₂. In one embodiment, at least a portion of the interior of the housing is coated with a photocatalyst. In the apparatus, each receiving member can independently receive a ultraviolet emitting light source that is either UV-V light or UV-C light. Furthermore, the number of ultraviolet emitting light sources can be one or two. In addition, a portion of an inner surface of the housing can comprise a reflective material. Furthermore, the light source that serves as a baffle can have a spiral or coil-like shape. In addition, the apparatus can include a sensor for measuring the concentration level of one or more contaminants in the air.

In yet a further aspect, there is provided an air purification apparatus comprising: a) a housing that defines an interior chamber, the housing having an inlet and an outlet; b) one or more ultraviolet emitting light emitting sources positioned in the chamber, each light source independently selected from the group consisting of UV-V light and UV-C light; c) a baffle positioned within the inner chamber providing resistance to airflow, the baffle being continuous throughout a length of the inner chamber; d) a photocatalyst coated either onto: (i) a portion of a surface of the baffle; (ii) a portion of an inner surface of the housing; or (iii) a combination thereof; and e) one or more anti-compression supports within the housing; wherein air enters the chamber through the inlet, travels through the inner chamber, and exits via the outlet. The photocatalyst can be, but is not limited to, TiO₂. Finally, the number of ultraviolet emitting light emitting sources can be one or two.

As air is forced through the air purification apparatus, most will traverse the chamber through unobstructed portions of the chamber; however, a portion of the air will be directed toward the baffle. This air will follow the baffle to traverse through the chamber, where it will eventually leave the chamber through the outlet. The portion of air that travels through the chamber along the baffle will reside in the chamber for a longer period of time than the air that passes straight through the chamber, and therefore, will be exposed to additional UVGI and PCO.

The foregoing summarizes the principal features of the apparatus and a few optional aspects. Wherever ranges of values are referenced within this specification, sub-ranges therein are intended to be included unless otherwise indicated. Where characteristics are attributed to one or another variant, unless otherwise indicated, such characteristics are intended to apply to all other variants where such characteristics are appropriate or compatible with such other variants.

### BRIEF DESCRIPTION OF THE DRAWINGS

Embodiments will now be described in detail with reference to the Drawings in which:

FIG. 1 is a cutaway perspective view of an embodiment of the air purification apparatus;

FIG. 2 is a cross sectional side view of the air purification apparatus shown in FIG. 1;

FIG. 3 is an elevational view of an end of the air purification apparatus shown in FIG. 1;

FIG. 4 is an elevational view of the opposing end of the air purification apparatus shown in FIG. 1;

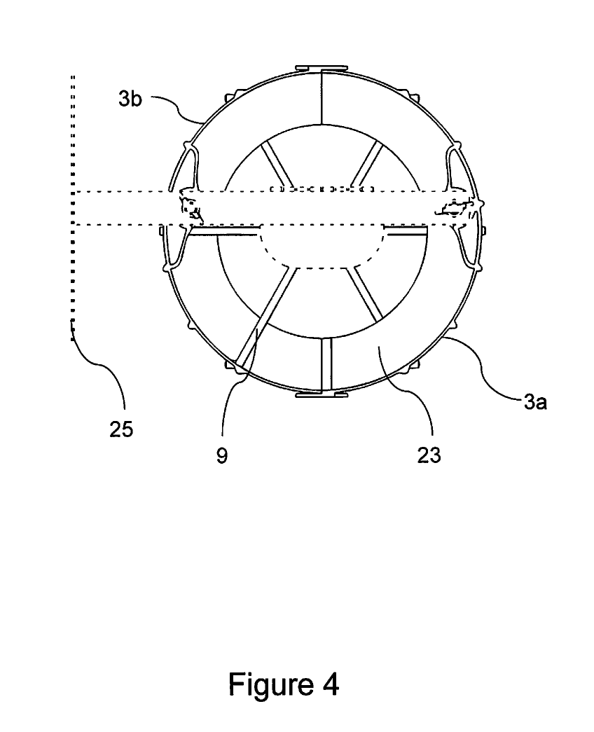

FIG. 5 is a top plan view of the air purification apparatus shown in FIG. 1;

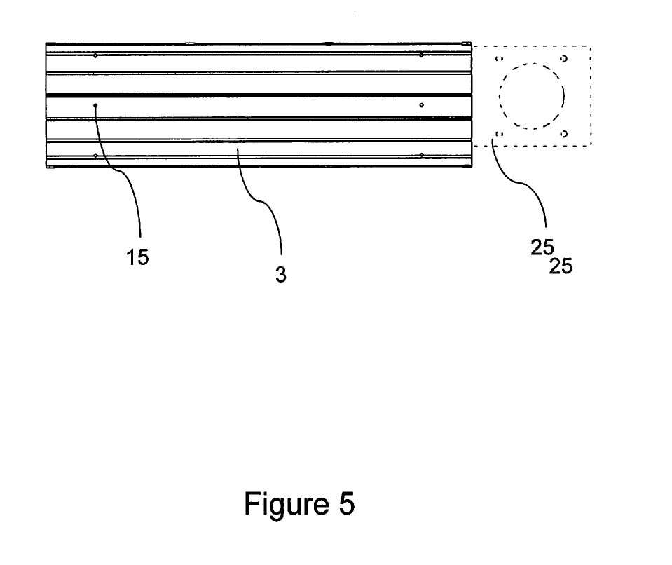

FIG. 6 is a side elevational view of the air purification apparatus shown in FIG. 1;

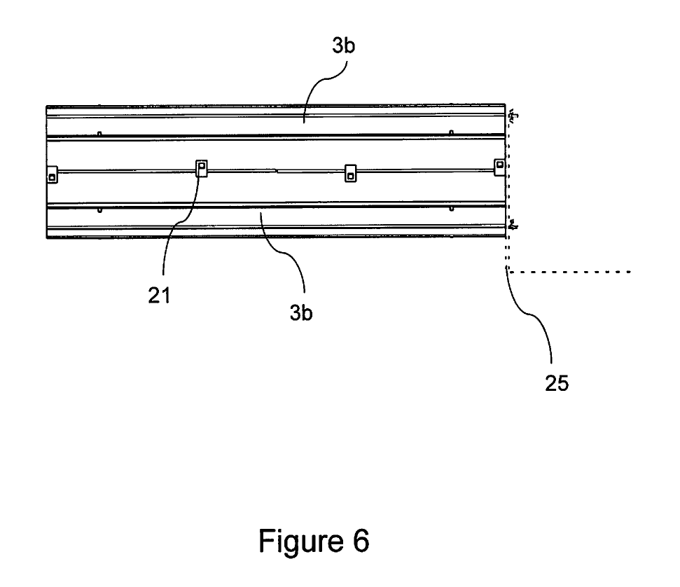

FIG. 7 is an exploded view of an embodiment of the air purification apparatus.

### DETAILED DESCRIPTION

The following is given by way of illustration only and is not to be considered limitative. Many apparent variations are possible without departing from the scope thereof.

An air purification apparatus 1, defined by an elongated housing 3, is shown in the Figures. The housing 3 has an inner surface 5a and an outer surface 5b and forms an interior chamber 7. Preferably, the housing 3 is tubular in design, however, other cross-sectional shapes other than circular, such as square or octagonal, are contemplated. The length and girth of the housing 3 may vary according to the threshold of purification that is required and/or the location where the air purification apparatus 1 is to be installed. For example, if the unit is to be installed in a small residential HVAC system, the dimensions of the housing 3 will match accordingly, whereas the dimensions of the housing 3 will be much larger if it is to be installed into a large commercial building. Materials for the housing 3 may vary, and may include such materials as aluminum, anodized aluminum or reflective metals. A majority of the decontamination and purification of the circulating air occurs in the chamber 7 formed by the housing 3. While it is preferred that the housing 3 forms a substantially airtight chamber 7, such may not be the case in other embodiments (for example, where multi-piece housings are use, or where housing apertures are present).

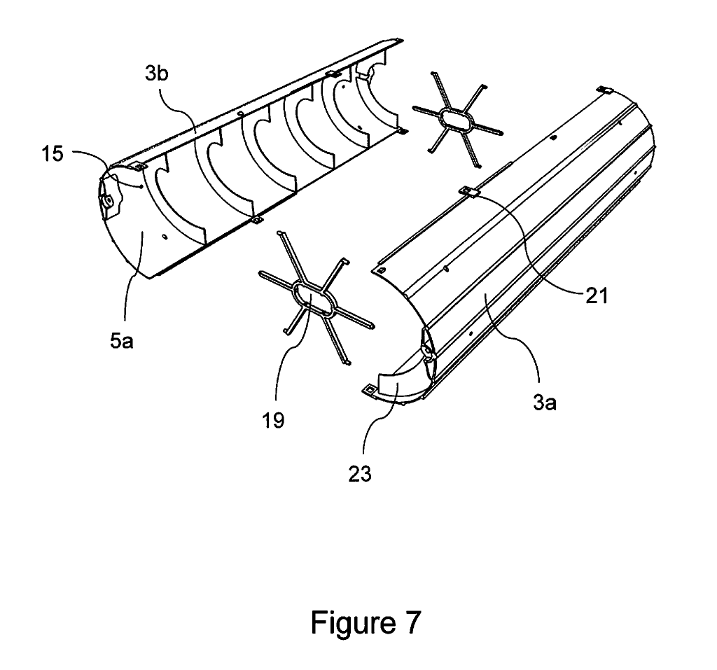

The housing 3 may be formed of a unitary piece of material, but may also be comprised of two or more pieces. For example, FIG. 7 illustrates a housing 3 comprised of a first housing piece 3a and a second housing piece 3b that mate to form the completed housing 3. In such a multi-piece configuration, the portions of the housing 3 have attachment means, such as a male-female snap means illustrated in the Figures, to secure the multiple pieces of the housing 3 together. Alternative attachment means for mating the multiple housing pieces 3a, 3b are contemplated, such as clips or nuts and bolts. The multi-piece housing 3 may also be permanently joined together during manufacture, such as by welding.

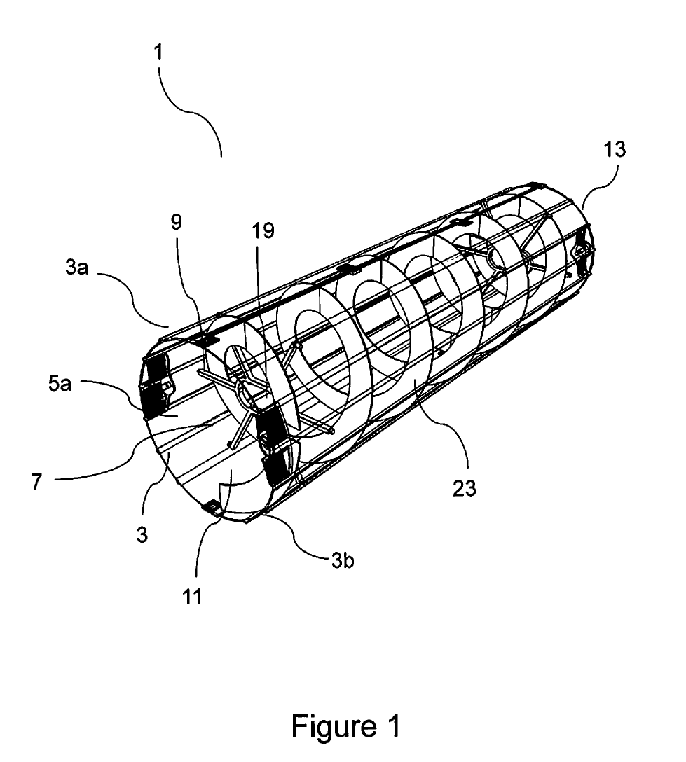

In one embodiment, additional strength, such as compressive strength, is provided to the housing 3 by the inclusion of anti-compression supports 9. The supports 9 may be placed at the inlet 11 and outlet 13 of the housing 3, but may also be placed along the length of the housing 3 as needed. The shape of the supports 9 is not limiting, and may have a star shaped appearance similar to those illustrated in FIGS. 1 and 7. In one embodiment, the supports 9 are locked in place by a portion of the support 9 interconnecting with corresponding apertures 15 in the housing 3 (as shown in FIGS. 1 and 7). However, other means of securing the supports 9 are contemplated, such as by welding the supports 9 to the housing 3 or by pressure fitting the supports 9 into the housing 3. The supports 9 may also serve the purpose of positioning the light source 17 within the chamber.

According to an embodiment, the air purification apparatus 1 comprises at least one light source 17. The air purification apparatus 1 may be manufactured and assembled with the inclusion of the light source 17; however, in another embodiment, the air purification apparatus 1 is manufactured without the light source 17, and the light source 17 is added at a later date, such as during installation of the apparatus 1. The light source 17 will need to be changed periodically, and therefore, is removably attached to the housing 3. In one embodiment, the light source 17 extends down the length of the housing 3, and is preferably centrally located in the chamber 7. In such an embodiment, the support(s) 9 allow the light source 17 to pass, such as by having an opening 19 that is able to accommodate the light source 17.

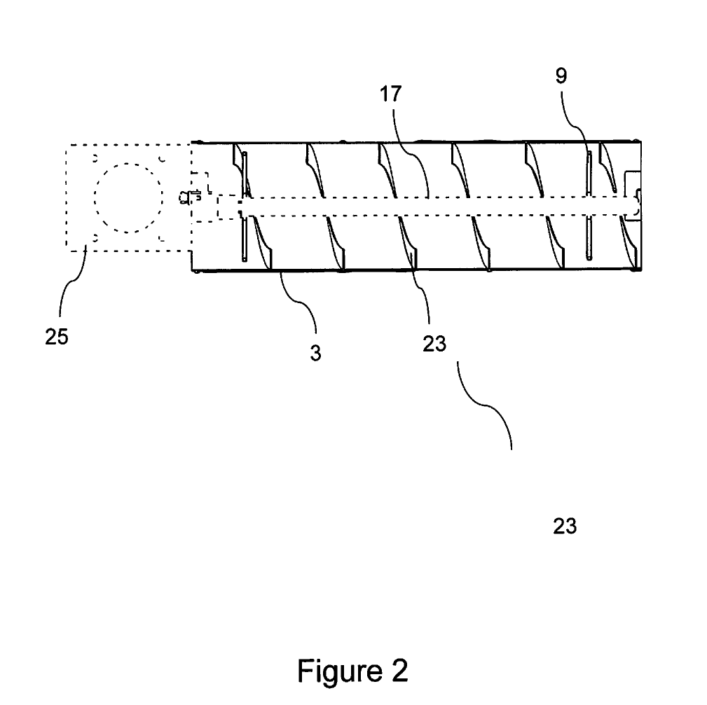

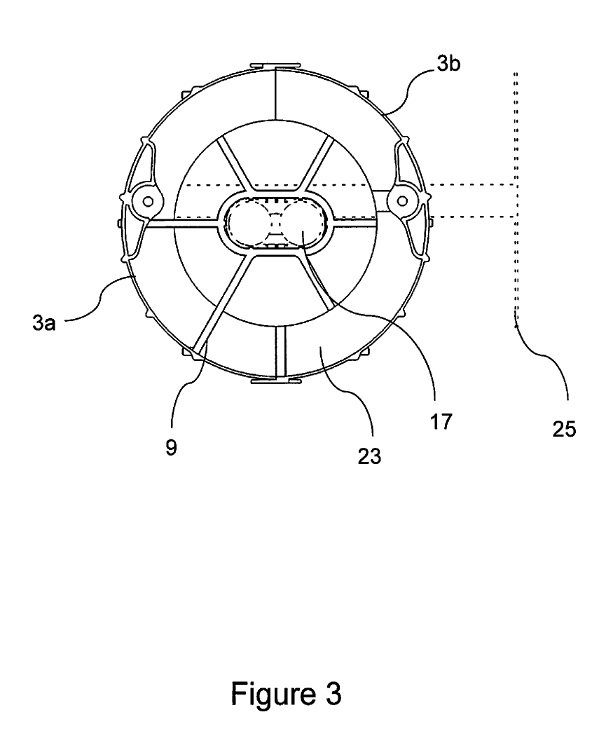

In order to provide ultraviolet germicidal irradiation and photocatalytic oxidation properties to the air purification apparatus 1, the light source 17 emits light at an appropriate wavelength. Preferably, the light source 17 emits ultraviolet C (UV-C) light, which typically corresponds with light having a wavelength in the 100 nm to 280 nm range. In some embodiments, ultraviolet V (UV-V) light, which typically corresponds with light having a wavelength in the 100 nm to 200 nm range, is utilized in the air purification apparatus 1 either in addition to UV-C light or as the sole light source 17. An exemplary air purification apparatus 1 with a light source 17 (shown in stippled lines) installed therein is illustrated in FIG. 2. According to one embodiment, the air purification apparatus 1 comprises more than one light source 17. For example, an air purification apparatus 1 with two light sources 17 (shown in stippled lines) is illustrated in FIG. 3. The addition of additional light sources 17 in the air purification apparatus 1 will increase the emission of light within the housing 3, or would allow for the inclusion of both a UV-C and UV-V light source 17 within the housing 3.

According to one embodiment of the invention, at least a portion of the inner surface of the housing 3 has a reflective surface. The reflective surface will reflect the light within the housing 3, propagating, directing and concentrating the ultraviolet light. For example, the inner surface of the housing 3 may be comprised of a reflective material, such as aluminum, or alternatively, the inner surface may be coated with a reflective compound, such as metallic paint.

In another embodiment, the housing 3 comprises a structure that provides resistance to the flow of air, such as a baffle 23. The structure may also provide a pathway that a portion of the air may follow to traverse the chamber 7. In one embodiment, the baffle 23 is in the outer portion of the chamber 7 and extends inwardly from the housing 3 into the chamber 7. According to one embodiment, the baffle 23 forms a continuous structure that extends along at least a portion of the chamber 7. Preferably, the baffle 23 forms e.g. a spiral or coil shape, as illustrated in the Figures, although other shapes, such as stepwise baffles 23, are contemplated. For example, if the housing 3 had a square or hexagonal cross section, the baffle 23 could extend along at least a portion of the chamber 7 in a spiral-like fashion, but as it would be shaped and dimensioned to extend from and communicate with the interior surface of the housing 3, it would not have a circular cross section.

According to another embodiment, the baffle 23 may be centrally located along the length of the housing. For example, the baffle 23 forms a spiral or coil shape and extends down the middle of the chamber 7. In such an embodiment, the baffle 23 could be attached to and supported by the inner surface 5a of the housing 3, but may also be attached to and supported by the housing supports 9. In such an embodiment, the baffle 23 would be close to a centrally located light source 17 and may even intimately contact the light source 17.

According to another embodiment, the chamber 7 comprises multiple baffles 23. The multiple baffles 23 may be of the same or dissimilar types and shapes. For example, a first portion of the chamber 7 near the inlet 11 comprises a first spiral baffle 23 that extends from the inner surface 5a of the housing; at a second portion of the chamber 7, the first spiral baffle 23 is discontinued and as second spiral baffle 23 that is centrally located in the chamber 7 commences; at a third portion of the chamber 7 near the outlet, the second spiral baffle 23 is discontinued, and a third spiral baffle 23 that extends from the inner surface 5a of the housing commences. Alternatively, the chamber 7 comprises a spiral baffle 23 that extends along the length of the housing 3, being either centrally located or extending from the inner surface 5a of the housing, where the baffle 23 is discontinuous, i.e. the baffle 23 has at least one gap along its length. The presence of at least one gap along the length of the baffle 23 effectively provide for multiple baffles 23 in the chamber 7.

With the incorporation of a baffle 23 in the housing 3, much of the circulating air will be forced into the baffle-free portion of the chamber 7 in order to pass through the housing with the least resistance; however, a portion of the air will be directed toward the baffle 23. For example, in the embodiment that comprises a spiraled baffle 23, centrally located or otherwise, a portion of the air that passing through the housing will be directed toward the baffle 23. This air will be able to follow the baffle 23 along its spiraled path to traverse through the chamber 7, where it will eventually leave the chamber 7 through the outlet. The portion of air that travels through the chamber 7 along the baffle 23 will reside in the chamber 7 for a longer period of time than the air that passes straight through the chamber 7, and therefore, will be exposed to additional UVGI and PCO.

The width of the baffle 23, i.e. the distance that the baffle 23 extends into the chamber 7, may vary, but cannot be such that the cross-sectional surface area of the chamber 7 is completely or substantially occluded, as this would not provide room for the light source 17. This may also slow down the air flow rate through the chamber 7 to unacceptable levels. Various embodiments having baffles 23 with differing widths are contemplated, as this will provide air purification apparatuses 1 that can accommodate a range of air flow rates.

The baffle 23 preferably extends substantially perpendicular with relation to the housing 3, however, the baffle 23 may also deviate from the normal. Deviation from the normal should still allow for the baffle 23 to provide some resistance in the interior chamber 7 of the housing 3, as well as be able to retain enough air to form a pathway through the housing 3. According to one embodiment, continuous baffles 23 are of a unitary construction, whereas in another embodiment, continuous baffles 23 are of a multi-piece construction. One example of a multi-piece construction of the baffle 23 is illustrated in FIG. 7. In this embodiment, the housing 3 itself is of a multi-piece construction, and each portion of the housing 3 comprises a portion of the baffle 23, such that when the first and second housing pieces 3a, 3b are assembled and attached, the baffle 23 pieces combine to form a continuous baffle 23.

In another embodiment of the invention, in lieu of or in addition to a baffle 23, the light source 17 is shaped and dimensioned to form the structure that provides air resistance, and also forms a path for the air to utilize to traverse the inside of the chamber 7. In this embodiment, the light source 17, which may be one or a plurality of lamps, has a coiled or spiral shape and extends along the length of at least a portion of the chamber 7.

According to one embodiment, at least a portion of the inner surface 5a of the housing 3 is coated with a photocatalyst that enables PCO to occur in the chamber 7. The photocatalyst is preferably a metal oxide capable of surface oxidation, such as TiO₂. In one embodiment, the baffle 23 is coated with photocatalyst. The baffle 23 may be entirely coated with the photocatalyst, however, it is contemplated that only a portion of the baffle 23 is coated. Coating the baffle 23 with the photocatalyst will increase the photocatalyst-coated surface area inside the chamber 7. In such an embodiment, the inclusion of a spiral or coil shaped baffle 23 or multiple baffles 23 will result in an increased photocatalyst-coated surface area in the housing 3 when compared to, for example, a linear or two-dimensional baffle 23.

According to one embodiment of the invention, the housing 3 of the air purification apparatus 1 has attachment points or brackets 25, as illustrated in the Figures, to allow for mounting of the air purification apparatus 1. The means of mounting the air purification apparatus 1 is not limiting, and typically will vary depending upon size and intended destination of the apparatus 1, and therefore can be left up to one of skill in the art.

In another embodiment of the invention, the housing 3 includes a sensor (not shown). The sensor measures the level of various contaminants within the chamber 7. In one embodiment, the sensor is connected to on/off controls of the air purification apparatus 1. If the level of at least one predetermined contaminant is above a certain threshold, then the air purification apparatus 1 is turned on. Alternatively, if the sensor detects that the level of at least one predetermined contaminant is below a certain threshold, the air purification apparatus 1 is turned off. This allows the air purification apparatus 1 to constantly monitor the air quality within the housing 3 and conserve energy when the operation thereof is deemed not necessary. Alternatively, the sensor measures air flow within the chamber 7, such as a pressure sensor, and once the air flow reaches a certain threshold, the air purification apparatus 1 turns on. This will allow the apparatus 1 to synchronize with, for example, an HVAC fan.

### CONCLUSION

The foregoing has constituted a description of specific embodiments. These embodiments are only exemplary. The invention in its broadest, and more specific aspects, is further described and defined in the claims which now follow.

These claims, and the language used therein, are to be understood in terms of the variants which have been described. They are not to be restricted to such variants, but are to be read as covering the full scope as is implicit and the disclosure that has been provided herein.

1. An air purification apparatus comprising:
a) a housing that defines an interior chamber, the housing having an inlet and an outlet;
b) one or more ultraviolet emitting light sources positioned within the interior chamber; and
c) one or more baffles providing resistance to airflow,
wherein air enters the chamber through the inlet, travels through the inner chamber, and exits via the outlet.

2. The air purification apparatus of claim 1, further comprising a photocatalyst.

3. The air purification apparatus of claim 2, wherein the photocatalyst is TiO₂.

4. The air purification apparatus of claim 1, wherein the number of ultraviolet emitting light sources is one or two.

5. The air purification apparatus of claim 1, wherein a portion of an inner surface of the housing comprises a reflective material.

6. The air purification apparatus of claim 1, wherein each ultraviolet emitting light source is independently selected from the group consisting of UV-V light and UV-C light.

7. The air purification apparatus of claim 1 comprising one baffle which is continuous throughout a length of the chamber.

8. The air purification apparatus of claim 1, further comprising a sensor for measuring a concentration level of one or more contaminants in the air.

9. An air purification apparatus comprising:
a) a housing that defines an interior chamber, the housing having an inlet and an outlet;
b) one or more ultraviolet emitting light source receiving members, each receiving member positioned within the inner chamber; and
c) one or more baffles providing resistance to airflow,
wherein air enters the chamber through the inlet, travels through the inner chamber, and exits via the outlet.

10. The air purification apparatus of claim 9, further comprising a photocatalyst.

11. The air purification apparatus of claim 10, wherein the photocatalyst is TiO₂.

12. The air purification apparatus of claim 9, wherein a portion of an inner surface of the housing comprises a reflective material.

13. The air purification apparatus of claim 9 comprising one baffle which is continuous throughout a length of the chamber.

14. An air purification apparatus comprising:
a) a housing that defines an interior chamber, the housing having an inlet and an outlet; and
b) one or more ultraviolet emitting light sources positioned within the inner chamber, with at least one ultraviolet emitting light source serving as a baffle to provide resistance to airflow through the interior chamber,
wherein air enters the chamber through the inlet, travels through the inner chamber, and exits via the outlet.

15. The air purification apparatus of claim 14, further comprising a photocatalyst.

16. The air purification apparatus of claim 15, wherein the photocatalyst is TiO₂.

17. The air purification apparatus of claim 14, wherein a portion of an inner surface of the housing comprises a reflective material.

18. The air purification apparatus of claim 14, wherein each ultraviolet emitting light source is independently selected from the group consisting of UV-V light and UV-C light.

19. An air purification apparatus comprising:
a) a housing that defines an interior chamber, the housing having an inlet and an outlet;
b) one or more ultraviolet emitting light emitting sources positioned in the chamber, each light source independently selected from the group consisting of UV-V light and UV-C light;
c) a baffle positioned within the inner chamber providing resistance to airflow, the baffle being continuous throughout a length of the inner chamber;
d) a photocatalyst coated either onto: (i) a portion of a surface of the baffle; (ii) a portion of an inner surface of the housing; or (iii) a combination thereof; and
e) one or more anti-compression supports within the housing;
wherein air enters the chamber through the inlet, travels through the inner chamber, and exits via the outlet.

20. The air purification apparatus of claim 19, wherein the photocatalyst is TiO₂.

21. The air purification apparatus of claim 19, comprising one ultraviolet emitting light emitting source.

22. The air purification apparatus of claim 19, comprising two ultraviolet emitting light emitting sources.
